(19) United States
(12) Patent Application Publication
(10) Pub. No.: US 2011/0202048 A1
(43) Pub. Date: Aug. 18, 2011

(54) METHODS FOR PAIN REDUCTION WITH FUNCTIONAL THERMAL STIMULATION AND TISSUE TREATMENT SYSTEMS

(75) Inventor: Dragan Nebrigic, Carlsbad, CA (US)

(73) Assignee: Solta Medical, Inc., Hayward, CA (US)

(21) Appl. No.: 12/823,214

(22) Filed: Jun. 25, 2010

Related U.S. Application Data
(60) Provisional application No. 61/303,739, filed on Feb. 12, 2010.

(51) Int. Cl. A61B 18/02; A61F 7/00

(52) U.S. Cl. 606/22; 607/100

(57) ABSTRACT

Methods and systems for delivering electromagnetic energy to a patient's tissue with an attendant reduction in the pain experienced by the patient due to tissue heating. Electromagnetic energy is delivered from an electrode through a skin surface to heat a first region of tissue beneath the skin surface and a second region of the tissue between the first region and the skin surface. During the delivery of the electromagnetic energy, a temperature of the second region of the tissue is caused to oscillate between at least one maximum temperature and at least one minimum temperature. The temperature oscillation may be induced by a timed sequence of cryogen pulses delivered to the electrode, which in turn extracts thermal energy out of the second region of tissue through the contacting skin surface.

### CROSS-REFERENCE TO RELATED APPLICATIONS

This application claims the benefit of U.S. Provisional Application No. 61/303,739, filed Feb. 12, 2010, the disclosure of which is hereby incorporated by reference herein in its entirety.

### BACKGROUND

The invention generally relates to methods and systems for treating tissue with electromagnetic energy and, more particularly, relates to methods and systems for reducing patient pain with functional thermal stimulation while treating tissue with electromagnetic energy.

Various cosmetic tissue treatments use energy delivery devices to non-invasively and non-ablatively treat tissue in order to improve a patient's appearance, such as smoothing and tightening skin, contouring along the jaw line and under the chin improving skin texture, softening wrinkles around the mouth, eyes and forehead, reducing cellulite, or removing skin spots or hair. These non-invasive, transcutaneous procedures involve no surgery or injections. Such non-invasive energy delivery devices emit electromagnetic energy in different regions of the electromagnetic spectrum to accomplish the tissue treatment with a reduced patient recovery time in comparison with ablative procedures.

Skin is a type of body tissue that includes plural distinct layers. The epidermis constitutes the visible outer layer on the surface. The dermis, which underlies the epidermis, contains collagen fibers, blood vessels, hair follicles, and other skin components. The hypodermis or subcutaneous fat layer, which underlies the dermis, consists of fat tissue and a web of collagen fibers in the form of fibrous septae running through the fat. The fibrous septae secure the dermis to the underlying bone and muscle. Collagen fibers are recognized to be a very flexible and stretchable protein and are characterized by a high tensile strength.

Wrinkles are mostly associated with the effects of advancing age and skin damage arising from exposure to damaging environmental factors. Collagen deteriorates and loses its elasticity, which results in the formation of rhytids or wrinkling of the epidermis. Environmental factors include sun damage from exposure to sunlight, air pollution, smoking, repetitive facial movements such as frowning, and the natural effects of gravity, which cause sagging of the skin with advancing aging. The occurrence of wrinkles is an unavoidable natural process.

Electromagnetic radiation, specifically light and heat, applied to the different layers of the skin can have a physiological effect on the skin's appearance. In particular, electromagnetic energy can arrest the formation of wrinkles and impart a more youthful skin appearance. High frequency treatment devices, such as radio-frequency (RF)-based treatment devices, may be used to treat skin tissue non-ablatively and non-invasively with heat. Such high frequency devices operate by transmitting high frequency energy through the epidermis to the underlying tissue, while actively cooling the epidermis to prevent thermal damage to a depth of the skin tissue near the skin surface. The high frequency energy heats the tissue at depths beneath the cooled region to a therapeutic temperature sufficient to denature the collagen, which causes the collagen fibers in the dermis to shrink and contract. In addition to the tightening of the treated tissue as the collagen fibers contract, treatment with high frequency energy also causes a mild inflammation. The inflammatory response of the treated tissue may cause new collagen to be generated over time, which can result in additional tissue contraction. When the inflammatory response of the treated tissue is highly significant, the new collagen formed is known as scar collagen.

Conventional high frequency treatment devices employ a handpiece, a disposable treatment tip coupled with a nose of the handpiece, and a high frequency generator connected by conductors inside the handpiece with an electrode in the treatment tip. Conventional electrodes consist of a pattern of one or more metallic features carried on a flexible electrically insulating substrate, such as a thin film of polyimide. The substrate contacts the patient's skin surface during treatment and the metallic features reside on the non-contact side of the substrate. The temperature of the treatment tip, which is measured by temperature sensors carried on the treatment tip, is correlated with the temperature of the patient's skin. During the procedure, the doctor controls the energy density of the high frequency power delivered from the electrode with a treatment setting. Treatment tips are frequently intended for single patient use and, therefore, are not reusable. Following the patient treatment, the doctor or treatment technician removes the treatment tip from the handpiece and, if disposable, discards it.

Patient pain is inherent in tissue treatments using electromagnetic energy. Patient pain is typically regulated to optimize the treatment result while also minimizing patient discomfort to make the procedure tolerable. A patient may be given an oral pain medication and/or a local topical anesthesia cream may be applied as a numbing agent. At the inception of the treatment procedure, the doctor will initially administer the high frequency energy at a treatment setting to one or more test sites and monitor patient feedback on the heat sensation associated with the treatment setting being used. A tolerable, yet comfortable, treatment setting for the treatment procedure is established based upon the patient feedback from the test sites.

The treatment electrode used in the treatment includes a conductor region delimited by an outer peripheral edge. For monopolar energy delivery, an edge effect has been observed at the outer peripheral edge that causes the energy density of the high frequency energy delivered to the tissue to be non-uniform across the surface area of the treatment electrode. Specifically, the energy density is highest near the peripheral edge of the electrode. As a result, tissue proximate to the outer peripheral edge of the electrode is heated to a higher temperature than tissue proximate to the interior surface area of the electrode. The higher temperatures near the peripheral edge form hot spot thermal zones that are a highly likely source of heat-related pain perceived by the patient. Because patient discomfort is linked with the treatment setting, reducing the treatment level to counteract the edge effect effectively reduces the average energy density for the high frequency energy delivered during the treatment procedure.

In general, the results and the chance for pain or discomfort scale with the treatment setting used by the doctor. What is needed, therefore, are systems and methods for reducing the pain associated with such tissue treatments so that patient discomfort is alleviated and therapeutic results can be improved by increasing the treatment setting.

### SUMMARY OF THE INVENTION

The invention is generally directed to systems and methods that deliver electromagnetic energy to transcutaneously treat tissue underlying a skin surface with electromagnetic energy, particularly during non-invasive and non-ablative therapeutic tissue treatments.

In one embodiment, a method is provided for operating an apparatus to treat a patient with electromagnetic energy. The method includes delivering the electromagnetic energy through a skin surface to heat a first region of tissue beneath the skin surface and a second region of the tissue between the first region and the skin surface. During the delivery of the electromagnetic energy to the first and second regions, a temperature of the second region of the tissue is caused to oscillate between at least one maximum temperature and at least one minimum temperature colder than the at least one maximum temperature. The oscillation of the temperature occurs at a given frequency effective to reduce pain associated with the delivery of the electromagnetic energy.

The temperature oscillation of the second region of the tissue may be caused by, while continuously heating the first and second regions with the electromagnetic energy, extracting thermal energy from the second region over intervals spaced apart in time to intermittently cool the second region. The extraction of the thermal energy may occur at a given frequency effective to reduce pain associated with the delivery of the electromagnetic energy or may be periodic at a given frequency effective to reduce pain associated with the delivery of the electromagnetic energy.

The delivery of the electromagnetic energy may include contacting a portion of a treatment electrode with the skin surface and, while maintaining the contact between the portion of the treatment electrode and the skin surface, delivering the electromagnetic energy. The temperature oscillation of the second region of the tissue may be caused by supplying a plurality of discrete amounts of a cryogen in a timed sequence as a series of cryogen pulses effective to intermittently cool the treatment electrode, and cooling the second region by heat transfer from the second region through the skin surface to the portion of the treatment electrode in contact with the skin surface. The timed sequence for the cryogen pulses may be selected to periodically cool the treatment electrode at a given frequency effective to reduce pain associated with the delivery of the electromagnetic energy, and the cryogen pulses may be supplied in an overlapping temporal relationship with the delivery of the electromagnetic energy. At least one of the cryogen pulses may be delivered before the delivery of the electromagnetic energy begins, and at least one of the cryogen pulses may be delivered during the delivery of the electromagnetic energy.

The at least one maximum temperature may include a plurality of maximum temperatures that increase with increasing time during the delivery of the electromagnetic energy to the first and second regions. The at least one minimum temperature may include a plurality of minimum temperatures that increase with increasing time during the delivery of the electromagnetic energy to the first and second regions.

In another embodiment, a method is provided for method operating an apparatus having a treatment electrode. The method includes delivering electromagnetic energy from the treatment electrode and, during the delivery of the electromagnetic energy, causing a temperature of the treatment electrode to oscillate between at least one maximum temperature and at least one minimum temperature colder than the at least one maximum temperature.

The oscillating temperature of the treatment electrode may be cause by supplying a plurality of discrete amounts of a cryogen in a timed sequence as a series of cryogen pulses that intermittently cool the electrode. The cryogen pulses may be timed to periodically cool the treatment electrode at a given frequency. The cryogen pulses may be supplied in an overlapping temporal relationship with the delivery of the electromagnetic energy.

In yet another embodiment, a system is provided for transcutaneously treating tissue located beneath a skin surface with electromagnetic energy. The system includes a cryogen supply configured to contain a cryogen, a handpiece including a valve coupled in fluid communication with the cryogen supply, a treatment tip attached in a removable manner with the handpiece, a generator configured to generate the electromagnetic energy, and a controller connected in electrical communication with the generator and with the valve. The treatment tip includes a treatment electrode configured to deliver the electromagnetic energy through the skin surface to the tissue and a nozzle coupled in fluid communication with the valve in the handpiece. The generator is electrically coupled by the handpiece with the treatment electrode. The controller is configured to cause the generator to deliver the electromagnetic energy to the treatment electrode for delivery through the skin surface to the tissue and configured to trigger the valve to deliver a plurality of discrete pulses each containing an amount of the cryogen to the nozzle during delivery of the electromagnetic energy.

The nozzle may be configured to deliver the cryogen to the treatment electrode, and the treatment electrode may include a portion configured to contact the skin surface. The controller may be configured to trigger the valve at a series of times to deliver the discrete pulses of the cryogen from the nozzle during delivery of the electromagnetic energy.

### BRIEF DESCRIPTION OF THE DRAWINGS

FIG. 1 is a diagrammatic view of a treatment system with a handpiece, a treatment tip, a control system, and a generator in accordance with an embodiment of the invention.

FIG. 2 is an exploded view of the treatment tip of FIG. 1.

FIG. 2A is a view of the backside of the treatment electrode.

FIG. 3 is a diagrammatic cross-sectional view of the treatment tip of FIGS. 1 and 2.

FIG. 3A is a cross-sectional view of a portion of the treatment tip in contact with the skin surface.

FIG. 4 is a graphical view showing the delivery of high frequency power to the patient's tissue and the delivery of coolant to the backside of the treatment electrode.

FIG. 5 is a diagrammatic view shown the temperature profile of a region of tissue below the skin surface.

### DETAILED DESCRIPTION

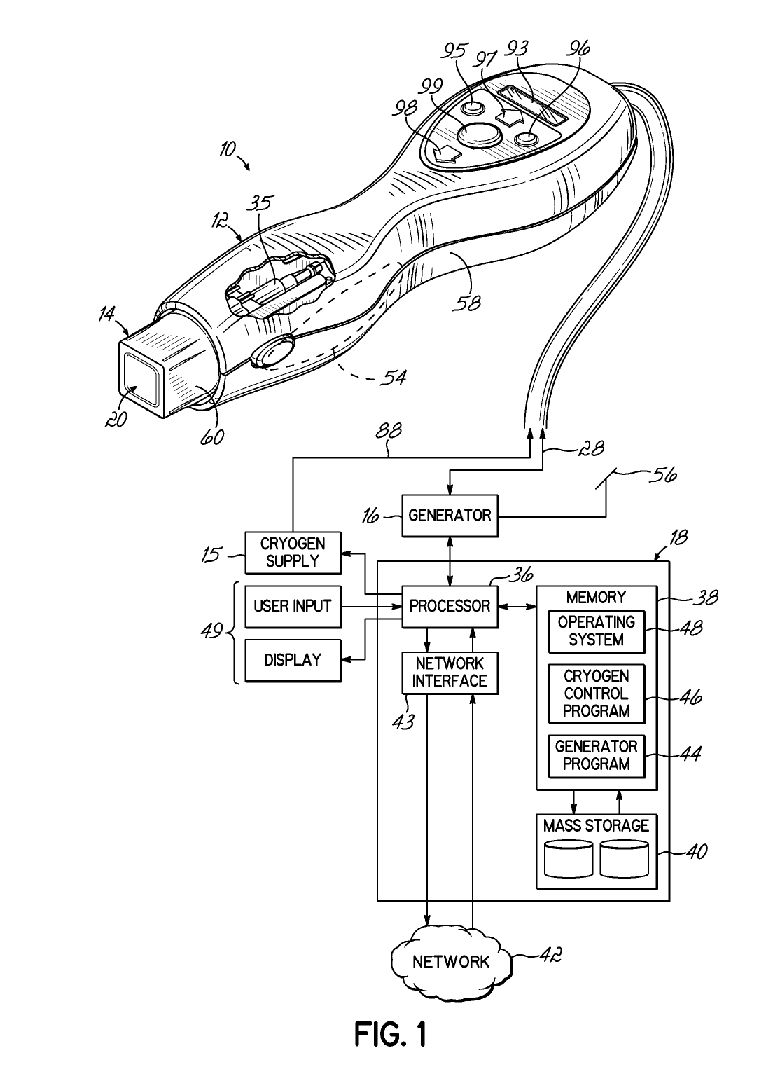

With reference to FIGS. 1, 2, 2A, 3 and 3A, a treatment apparatus 10 includes a handpiece 12, a treatment tip 14, a cryogen supply 15, a generator 16, and a system controller 18. The treatment tip 14 is coupled in a removable and releasable manner with the handpiece 12. The treatment tip 14 carries an electromagnetic energy delivery member in the representative form of a treatment electrode 20. In a representative embodiment, the treatment electrode 20 may include an electrically-insulating substrate 22 composed of a non-conductive dielectric material and a region 24 of an electrical conductor carried on the electrically-insulating substrate 22. In one embodiment, the substrate 22 of the treatment electrode 20 may comprise a thin flexible base polymer film carrying the conductor region 24 and conductive (e.g., copper) traces or leads 25 on the substrate 22 that are used to electrically couple the conductor region 24 with the generator 16 and temperature sensors 52 with the system controller 18. The base polymer film of substrate 22 may be, for example, polyimide or another material with a relatively high electrical resistivity and a relatively high thermal conductivity. Instead of the representative conductor region 24, the conductor region of treatment electrode 20 may be segmented into plural individual electrodes that can be individually powered to deliver electromagnetic energy to the tissue 30.

The conductor region 24 of the treatment electrode 20 is electrically coupled by a set of insulated and shielded conductors 28 that extend exteriorly of the handpiece 12 to the generator 16. The substrate 22 may also carry a non-volatile memory chip 27, such as an Erasable Programmable Read-Only Memory (EPROM), that retains its data when unpowered. The memory chip 27 is coupled by the conductive leads 25 with the system controller 18.

The generator 16, which has the representative form of a high frequency power supply, is equipped with a conventional electrical circuit operative to generate high frequency electrical current, typically in the radio-frequency (RF) band of the electromagnetic spectrum. The operating frequency of generator 16 may be in the range of 200 kHz to about 20 MHz. In one embodiment, the generator 16 is a 400 watt, 6.78 MHz high frequency generator. The electrical circuit in the generator 16 converts a line alternating current voltage into drive signals for the treatment electrode 20. The drive signals have an energy content and a duty cycle appropriate for the amount of power and the mode of operation that have been selected by the clinician, as understood by a person having ordinary skill in the art.

The system controller 18 is interfaced with the cryogen supply 15 and with the generator 16, and coordinates the operation of the treatment apparatus 10. In particular, the system controller 18 regulates the power delivered from the generator 16 to the treatment electrode 20 by setting the operational parameters of the generator 16 and by setting the operational parameters of the cryogen supply 15. Under the control of the system controller 18 and operator interaction with controls at the system controller 18 and handpiece 12, the treatment apparatus 10 is adapted to non-invasively and non-ablatively deliver electromagnetic energy in a high frequency band of the electromagnetic spectrum, such as the radiofrequency (RF) band, to an underlying region of a patient's tissue 30.

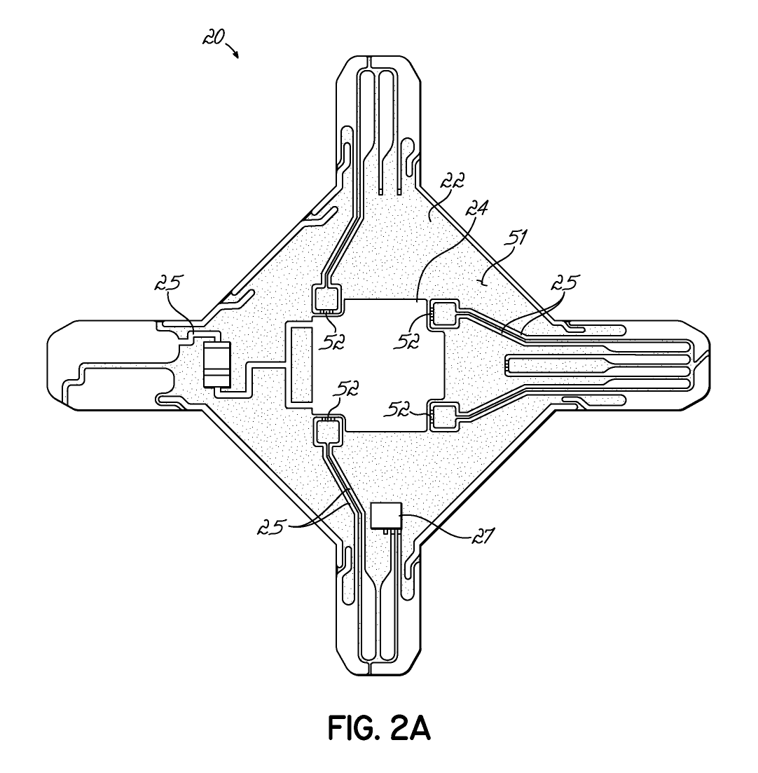

The electromagnetic energy imparts a therapeutic effect to heat tissue 30 in a targeted region 32 beneath the patient's skin surface 26, as best shown in FIG. 3A, to a therapeutic temperature. Because of the concurrent cooling from the skin surface 26 inward, a portion of the tissue 30 between region 32 and the skin surface 26 are heated to a non-therapeutic temperature such that this shallow tissue portion is not modified. The delivered energy volumetrically heats a region 32 of the tissue 30 to a targeted temperature range. The elevation in temperature within the heated region 32 may produce for example, changes in collagen in the tissue 30 that achieve a desired treatment result, such as removing or reducing wrinkles and otherwise tightening the skin to thereby improve the appearance of a patient receiving the treatment.

System controller 18 may represent practically any computer, computer system, or programmable device recognized by a person having ordinary skill in the art. System controller 18 typically includes at least one processor 36 coupled to a memory 38. Processor 36 may represent one or more processors (e.g., microprocessors), and memory 38 may represent the random access memory (RAM) devices comprising the main storage of system controller 18, as well as any supplemental levels of memory, e.g., cache memories, non-volatile or backup memories (e.g. programmable or flash memories), read-only memories, etc. In addition, memory 38 may be considered to include memory storage physically located elsewhere in system controller 18, e.g., any cache memory in processor 36, as well as any storage capacity used as a virtual memory, e.g., as stored on a mass storage device 40 or another computer (not shown) coupled to system controller 18 via a network interface 43 over a network 42. The system controller 18 operates under the control of an operating system 48, and executes or otherwise relies upon various computer software applications, components, programs, objects, modules, data structures, etc. (e.g., power modulation control program 44 or cryogen control program 46 executing in memory 38). The mass storage device 40 may store a copy of the generator control program 44 and a copy of the cryogen control program 46.

The system controller 18 also typically receives a number of inputs and outputs for external communications of information. For interface with a user or operator, the system controller 18 typically includes one or more user interface devices 49, such as input devices (e.g., a keyboard, a mouse, a trackball, a joystick, a touchpad, a keypad, a stylus, and/or a microphone, among others). Interface devices 49 may also include a display or other output device (e.g., a CRT monitor, an LCD display panel, and/or a speaker, among others). The interface to the system controller 18 may also be through an external terminal connected directly or remotely to system controller 18, or through another single (or multi) user computer (not shown) communicating with the system controller 18 via network 42, modem, or other type of communications device. Instructions delivered to the system controller 18 via the user interface devices 49 may be used to adjust the generator 16 to establish an arbitrary treatment setting. Information displayed by the user interface devices 49 may include the amount of energy delivered, tissue impedance, duration, and feedback to the operator relating to procedure technique. System controller 18 may optionally be linked with a nonvolatile memory (not shown) carried by the handpiece 12 or with a nonvolatile memory (not shown) carried by the treatment tip 14.

In general, the routines executed to implement the embodiments of the invention, whether implemented as part of an operating system or a specific application, component, program, object, module or sequence of instructions will be referred to herein as “computer program code”, or simply “program code”. The computer program code typically comprises one or more instructions that are resident at various times in various memory and storage devices in a computer, and that, when read and executed by one or more processors in a computer, causes that computer to perform the steps necessary to execute steps or elements embodying the various aspects of the invention. Moreover, while the invention has and hereinafter will be described in the context of fully functioning computers and computer systems, those skilled in the art will appreciate that the various embodiments of the invention are capable of being distributed as a program product in a variety of forms, and that the invention applies equally regardless of the particular type of computer readable media used to actually carry out the distribution. Examples of computer readable media include but are not limited to physical, recordable type media such as volatile and non-volatile memory devices, floppy and other removable disks, hard disk drives, optical disks (e.g., CD-ROM's, DVD's, etc.), among others, and transmission type media such as digital and analog communication links

During a non-ablative and non-invasive tissue treatment, a portion of one side or surface 50 of the treatment electrode 20 is placed into a directly contacting relationship with the skin surface 26 of the patient. The conductor region 24 of the treatment electrode 20 is physically carried on a non-contact side or surface 51 of the substrate 22 of the treatment electrode 20 and is therefore separated by the substrate 22 from the skin surface 26 (FIG. 3A). Hence, in the representative embodiment, the substrate 22 is arranged between the conductor region 24 and the skin surface 26. Electromagnetic energy is transmitted in a transcutaneous manner from the conductor region 24 on one side 51 of the substrate 22 through the thickness of substrate 22 across the area of the surface 50 registered with the conductor region 24 to the corresponding surface area of skin surface 26 and the underlying tissue 30 by capacitively coupling with the tissue 30.

The treatment tip 14 includes a plurality of sensors 52 that output readings that are used as feedback by the system controller 18 to control the treatment process. In one embodiment, the sensors are temperature sensors 52, such as thermistors or thermocouples, that are constructed to detect the temperature of the treatment electrode 20 and/or treatment tip 14. In the representative embodiment, the temperature sensors 52 are disposed on the surface 51. The measured temperature reflects the temperature of the treated tissue 30 and may be used as feedback in a control loop by the system controller 18 for controlling energy delivery and/or cooling of the skin surface 26. The handpiece 12 or treatment tip 14 may also include pressure sensors (not shown) for detecting physical contact between the treatment electrode 20 and the skin surface 26. In an alternative embodiment, one or more of the sensors 52 may be impedance sensors.

An activation button 54, which is accessible to the operator from the exterior of the handpiece 12, is configured to be actuated to close a switch in a normally open circuit with the generator 16. The closed circuit energizes the treatment electrode 20. Actuation of the activation button 54 triggers delivery of a dose of the high frequency energy over a short timed delivery cycle to the target tissue 30. After a lapsed treatment time, the delivery of high frequency energy from the treatment electrode 20 to the tissue 30 at the treatment site is discontinued and the handpiece 12 is manipulated to position the treatment tip 14 near a different treatment site on the skin surface 26. Another cycle is then initiated to deliver another dose of high frequency energy to the patient's tissue 30. The treat and move process is repeated for an arbitrary number of treatment sites distributed across the skin surface 26.

High frequency electrical current flowing between the treatment electrode 20 and the patient is concentrated at the skin surface 26 and within the underlying tissue 30 across the contacting surface area of the treatment electrode 20. Capacitive coupling of the high frequency electromagnetic energy relies on energy transfer through the dielectric material of the substrate 22 to create an electric field across the surface area where the treatment electrode 20 contacts the patient's body. The time-varying electric field induces electrical currents within the surrounding tissue 30 beneath the skin surface 26. Because of the natural resistance of tissue 30 to electrical current flow, volumetric heating results within the tissue 30. The volumetric heating delivers a therapeutic effect to the region 32 of the tissue 30 near the treatment site. For example, heating to a temperature of 60° C. or higher may contract collagen fibers and/or form nascent collagen within the region 32, which will result in tissue tightening or another aesthetic effect to improve the patient's appearance. The heating depth in the tissue 30 is based upon the size and geometry of the treatment electrode 20 and, contingent upon the selection and configuration of the treatment tip 14 and cooling with a reverse thermal gradient, can be controlled to extend from a few hundred microns beneath the skin surface 26 to several millimeters.

A non-therapeutic passive return electrode 56 is used to electrically couple the patient with the generator 16. During patient treatment, the high frequency current flows from the treatment electrode 20 through the treated tissue 30 and the intervening bulk of the patient to the return electrode 56 and then to the generator 16 through the conductors 22 to define a closed circuit or current path. The return electrode 56 is physically attached by, for example, adhesive to a site on the body surface of the patient, such as the patient's back. The surface area of the return electrode 56 in contact with the patient is relatively large in comparison with the surface area of the treatment electrode 20. Consequently, at the tissue adjacent to the return electrode 56, the current density flowing from the patient to the return electrode 56 is distributed across the larger surface area and is relatively low in comparison with the current density flowing from the treatment electrode 20 of smaller surface area to the patient. Because negligible heating is produced at its attachment site to the patient, a non-therapeutic effect is created in the tissue adjacent to the return electrode 56.

The system controller 18 includes digital and/or analog circuitry that couples the controller 18 in communication with the generator 16 for supplying control signals thereto and for receiving feedback information from sensors that is used in generating the control signals. Generator control program 44 resident as an application in the memory 38 is executed as an algorithm by the processor 36 in order to issue commands that control the operation of the generator 16.

Although the treatment electrode 20 and the return electrode 56 are representatively configured for the delivery of monopolar high frequency energy, the treatment electrode 20 may be configured to deliver bipolar high frequency energy. The modifications to the treatment apparatus 10 required to deliver bipolar high frequency energy are familiar to a person having ordinary skill in the art. For example, the return electrode 56 may be eliminated from the treatment apparatus 10 and a bipolar treatment electrode substituted for the monopolar treatment electrode 20.

With continued reference to FIGS. 1, 2, 2A, 3, and 3A, the handpiece 12 is constructed from an outer housing 58 and the treatment tip 14 includes an outer housing 60 that is mechanically coupled with the housing 58 to establish an assembly. The handpiece 12 and treatment tip 14 include complementary electrical/fluid interfaces (not shown) that are coupled together when the housings 58, 60 are mechanically coupled. The housings 58, 60 may be fabricated by an injection molding process using a suitable polymer resin as a construction material. The handpiece 12 has a smoothly contoured shape suitable for gripping and manipulation by an operator. The operator maneuvers the treatment tip 14 and treatment electrode 20 to a location proximate to the skin surface 26 and, typically, to place the treatment electrode 20 in a contacting relationship with the skin surface 26.

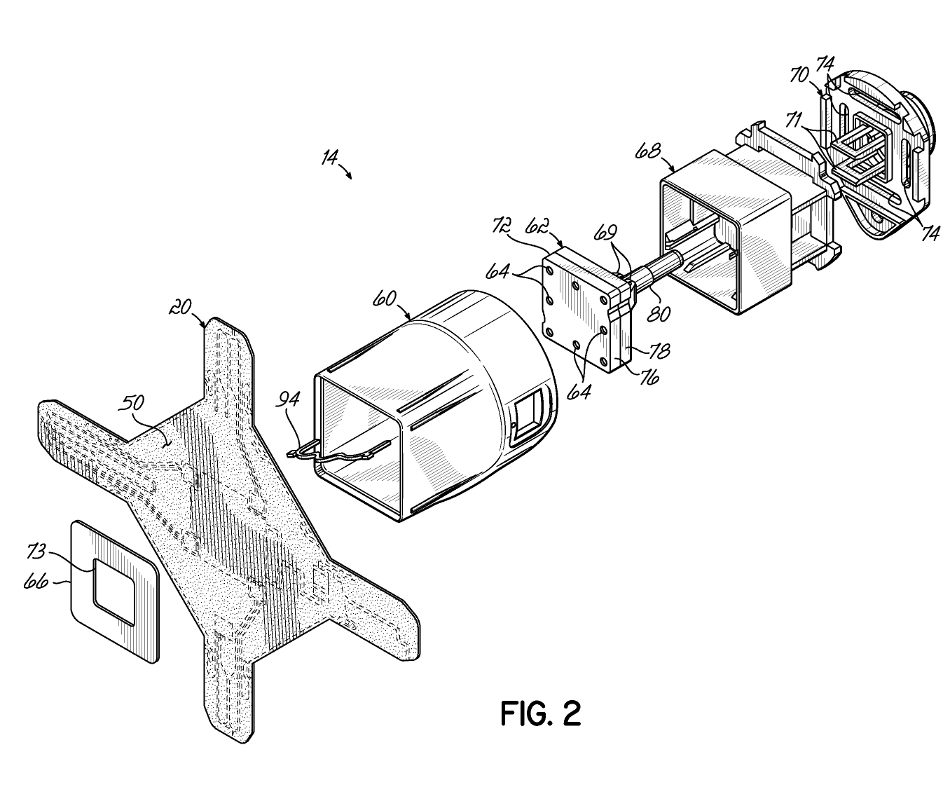

With reference to FIGS. 2 and 3, the treatment tip 14 includes the treatment electrode 20, the housing 60, a nozzle 62 that is configured with a head 72 having multiple orifices 64, a tip frame 66 that is coupled with the treatment electrode 20, and a pair of structural members 68, 70 that support the nozzle 62. The structural member 68, 70 are assembled with the nozzle 62 such that the head 72 of the nozzle 62 is recessed inside the similarly shaped hollow interior of the structural member 68. The assembly of the treatment electrode 20 and structural members 68, 70 is secured together by complementary pairs of clip fasteners 69, 71 respectively on the nozzle 62 and structural member 70. The treatment electrode 20, which is shown in an unfolded state, is wrapped about the exterior of the structural member 68 such that the leads 25 can be contacted through openings 74 defined in structural member 70. A bridge 94 provides backside mechanical support and rigidity to the flexible treatment electrode 20. An optional heat spreader (not shown) may be disposed between the head 72 and the treatment electrode 20. The tip frame 66 has a central aperture 73 that is registered with the conductor region 24 of the treatment electrode 20.

The nozzle 62 is an assembly that includes a spray plate 76, a flange 78 that is coupled with the spray plate 76 to define the head 72, and a stem 80 that extends rearwardly from the flange 78. Extending axially along the length of the stem 80 is a flow channel 82 with an inlet 84 and an outlet 86. Cryogen is pumped from the cryogen supply 15 through tubing 88 partially inside the handpiece 12 and mechanically coupled with the inlet 84 to the flow channel 82. The cryogen supply 15 may be a pre-filled canister containing a pressurized cryogen like the low boiling point fluids 1,1-Difluoroethane (R-152a refrigerant) or 1,1,1,2-Tetrafluoroethane (R-134a refrigerant). Disposed between the flange 78 and spray plate 76 is a system of flow channels 90 that distribute the cryogen to passages 92 extending through the thickness of the spray plate 76. Each of the passages 92 terminates at one of the orifices 64.

The handpiece 12 is equipped with a valve 35 that is used to deliver a metered amount of cryogen to the nozzle 62. The system controller 18 includes digital and/or analog circuitry that couples the controller 18 in communication with the valve 35 for supplying control signals thereto and for receiving feedback information from sensors that is used in generating the control signals. Cryogen control program 46 resident as an application in the memory 38 is executed as an algorithm by the processor 36 in order to issue commands that control the operation of the valve 35.

The cryogen is ejected from each of the orifices 64 in the nozzle 62 in a pulse as an atomized or non-atomized spray or stream of cryogen directed toward the backside 51 of the treatment electrode 20 and, in particular, toward the conductor region 24. The cryogen impinges and wets the backside 51 of the treatment electrode 20 and subsequently evaporates in a phase transition from liquid to gas, which extracts heat and produces the cooling effect. Because of the low thermal mass, the temperature of the treatment electrode 20 drops rapidly with cryogen evaporation. The treatment electrode 20 is colder than the tissue 30. The cooling effect from the reduced temperature is communicated through the substrate 22 to the skin surface 26 and into the tissue 30 to extract heat from the tissue 30. Cooling occurs by heat conduction across interface 20a that extracts thermal energy from the tissue 30 due to the temperature difference between the tissue 30 and the colder treatment electrode 20.

The cooling caused by the cryogen is superimposed on the heating profile from the delivered electromagnetic energy. Specifically, the cooling of tissue 30 competes with the volumetric heating from the electromagnetic energy such that a reverse thermal gradient is produced in tissue 30 and the therapeutic effect is delivered only to the region 32. Because thermal energy is extracted from the tissue surface 26 inward into the tissue 30, a region 31 of tissue 30 between region 32 and the skin surface 26 is cooled and, preferably, is prevented from reaching a therapeutic temperature. Region 31 is colder than region 32 and the temperature of region 31 increases in a temperature gradient from the skin surface 26 across the thickness of region 31. The temperature in the heating profile for region 31 is lowest at or near the skin surface, and is highest at or near the interface between regions 31, 32.

The handpiece 12 includes a display 93, controls 95, 96 that scroll different functions on the display 93, controls 97, 98 used to respectively increase and reduce the setting for the function currently on the display 93, and a control to engage a changed setting. The display 93 may be used to display information including, but not limited to, energy delivered, tissue impedance, duration, and feedback on procedure technique. The availability of the information displayed on the display 93 may conveniently eliminate the need to display identical information on the interface devices 49, or may duplicate displayed information by the interface devices 49. By displaying information at the handpiece 12, the operator can focus on the procedure without diverting his attention to glance at information displayed by the display on the interface devices 49. In one embodiment, the display 93 may constitute a thin, flat liquid crystal display (LCD) comprised of a light source or reflector and an arbitrary number of color or monochrome pixels arrayed in front of the light source or reflector. A driver circuit (not shown) is provided to control the operation of the display 93.

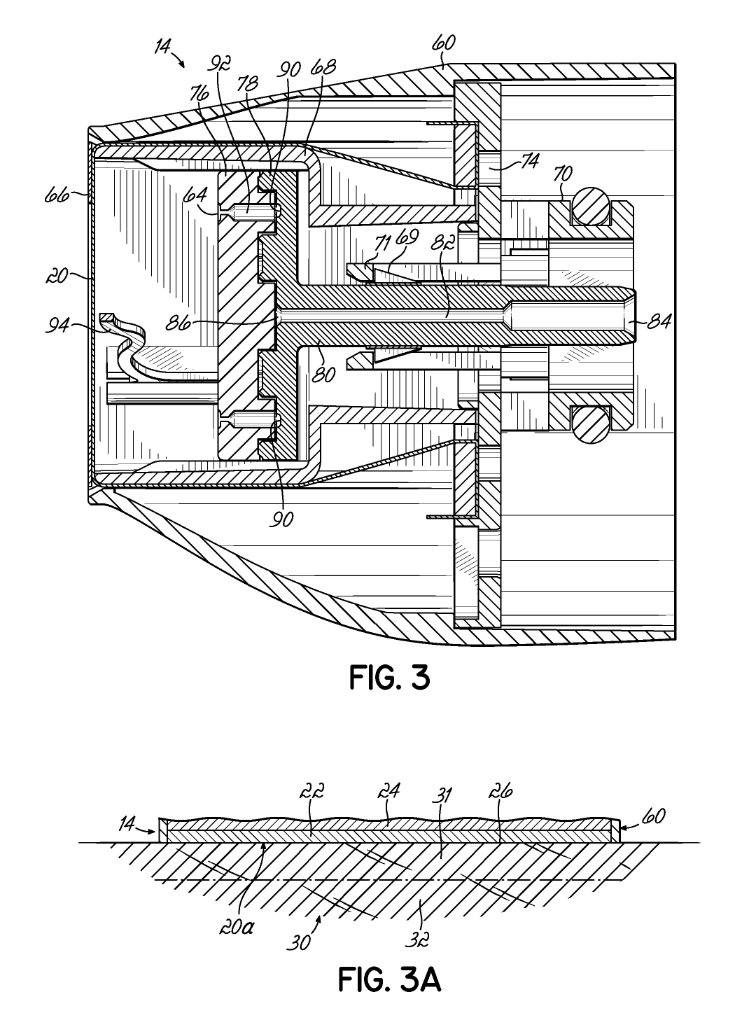

With reference to FIG. 4, a protocol for a treatment repetition is shown in which the tissue cooling is parsed into a plurality of individual and discrete pulses to reduce patient pain with functional thermal stimulation while treating tissue with high frequency energy. A power or treatment pulse 100 of electromagnetic energy is delivered from the generator 16 to the treatment electrode 20 at each treatment location on the skin surface 26 and delivered transcutaneously to the tissue 30 over a treatment time, T. An initial cryogen pulse 104, which occurs before the treatment pulse 100 is initiated to begin the delivery of electromagnetic energy, operates to pre-cool the tissue 30 at the treatment site by heat transfer from the epidermis to the treatment electrode 20. Typically, region 31 is held at a temperature that is below a therapeutic temperature in order to prevent harm during treatment.

Cryogen pulses 106, 108, 110 are sequentially delivered at intervals during the treatment time, T, and consequently while the electromagnetic energy is being delivered in treatment pulse 100 to the tissue 30. As a result, the high frequency energy delivered to the tissue 30 in power pulse 100 fails to heat the tissue 30 in region 31 proximate to the skin surface 26 to a temperature sufficient to cause significant thermal damage. The region 32 of tissue 30 that is not significantly cooled by pre-cooling will volumetrically warm up to therapeutic temperatures, which cause a desired therapeutic effect. The cooling creates a reverse thermal gradient in the tissue 30 such that the temperature of the tissue 30 in region 31 is cooler than the temperature of the tissue 30 in region 32 (FIG. 3A). After power delivery concludes at the end of treatment pulse 100, a train of additional cryogen pulses 112 is delivered to post-cool the tissue 30 at the treatment site. Post-cooling may prevent or reduce heat delivered deeper into the region 32 of the tissue 30 from conducting upward and heating the shallower tissue region 31 to temperatures which could cause thermal damage even though external energy delivery from treatment electrode 20 has ceased.

The treatment repetition is conducted by contacting the treatment electrode 20 with the skin surface 26, applying power to the treatment electrode 20 for the characteristic treatment time, T, and taking the protocol of FIG. 4 into account, and lifting the treatment electrode 20 from the skin surface after the cryogen pulses 112 are delivered. The amount and/or duration of cooling caused by cryogen pulses 104, 106, 108, 110, 112 may be used to select the depth of the protected region 31 of tissue 30 between the region 32 and the skin surface 26.

Each of the cryogen pulses 106, 108, 110 is triggered to be sprayed by the valve 35 onto the treatment electrode 20 at given times, t₁, t₂, t₃, respectively, after the onset of energy delivery at time t₀. The successive times, t₁, t₂, t₃, are measured relative to time t₀. Each of the pulses 106, 108, 110 is characterized by an amount of cryogen that is delivered over a given pulse width or time period, Δt. The amount of cryogen delivered and the time period determine a rate of cryogen delivery. The cryogen pulses 106, 108, 110 overlap temporally with the pulse 100 of electromagnetic energy and occur after the initiation of energy delivery at time, t₀, and before the conclusion of the treatment at treatment time, T. The temporal overlap occurs with the delivery of the cryogen pulses 106, 108, 110 coinciding in time with the delivery of electromagnetic energy in pulse 100.

The pulses 106, 108, 110 are temporally separated such that any continuity in cryogen delivery is interrupted by time gaps. A time gap equal to the difference in t₂ and (t₁+Δt) separates the triggering of pulse 108 from the conclusion of pulse 106. A time gap equal to the difference in t₃ and (t₂+Δt) separates the triggering of pulse 110 from the conclusion of pulse 108. The time gaps are substantially uniform so that the cryogen delivery is intermittent and periodic with a frequency given by the reciprocal of the time gap. The time gap between t₀ and t₁ for triggering pulse 106 is selected in conjunction with the time difference between the incidence of pulse 104 and the onset of energy delivery at t₀. The pulses 106, 108, 110 represent discrete amounts of cryogen delivered in a time sequence to intermittently cool the treatment electrode 20 and cause the temperature of the electrode 20 (and region 31) to intermittently increase and decrease. Preferably, the pulses 106, 108, 110 are delivered periodically to the treatment electrode 20.

The processor 36 of the system controller 18 executes the generator control program 46 resident as an application in the memory 38 in order to issue commands that control the operation of the generator 16 to provide the treatment pulse 100 of electromagnetic energy. Similarly, the processor 36 of the system controller 18 executes the cryogen control program 46 resident as an application in the memory 38 in order to issue commands that control the operation of the cryogen supply 15 and valve 35 to provide the cryogen pulses 104, 106, 108, 110, 112. The cryogen pulses 104, 106, 108, 110, 112 operate to systematically cool the tissue 30 between region 32 and the skin surface 26 to a non-therapeutic temperature during the course of the procedure. Energy delivery and coolant delivery are controlled and coordinated by operation of the system controller 18 in conjunction with user input via user interface devices 49 at the system controller 18, controls 96-99 at the handpiece 12, and software settings in the generator and cryogen control programs 44, 46.

Parameters in the generator control program 44 and cryogen control program 46 are adjusted to implement the pattern of cryogen pulses 104, 106, 108, 110, 112 that overlap temporally with treatment pulse 100 for each treatment repetition as shown in FIG. 4. The current limit for the high frequency power in pulse 100 may be set to a value in the range of about 0.3 amperes to about 1.6 amperes with 0.01 ampere adjustment increment, which varies according to the treatment and is specific to the type of treatment tip 14. The maximum energy permitted to be delivered by a power pulse during a repetition may range from ten (10) Joules to 300 Joules with a precision of one (1) Joule using electrodes having a size ranging from one (1) cm² to twenty (20) cm².

The duration of the high frequency power pulse 100 may be set within a range of 200 milliseconds to 300 milliseconds with an adjustment increment of 10 milliseconds. The delivery of the high frequency energy may be constrained by the current limit selection and the maximum power selection. The selected maximum power is usually reached within an initial fraction of the pulse duration and is stabilized by a Proportional-Integral-Derivative (PID) feedback loop. In addition, the current-limiting mode based upon the selected current limit may begin to function only after the target power has been reached, and takes an additional fraction of the pulse duration to stabilize the current delivery. Hence, the square pulse shape for the pulse 100 is diagrammatically ideal and ignores initial transient effects during delivery.

The duration of each of the cryogen pulses 106, 108, 110, which overlap temporally with the power pulse 100 of high frequency energy, may be set within a range of two (2) milliseconds to forty-nine (49) milliseconds with an adjustment increment of one (1) millisecond. This range results in a frequency for the oscillating cooling that is in a range of about 20 Hz to 500 Hz. For a duration of 200 milliseconds for the high frequency power pulse 100, as a numerical example, the number of cryogen pulses supplied during the delivery of electromagnetic would range from four (4) to 100 depending on the specific cryogen pulse frequency. The number of cryogen pulses in the protocol for a treatment repetition may differ from the number of pulses 104, 106, 108 shown in FIG. 4. In addition, the amount of cryogen per pulse and/or the pulse width may differ among the different pulses 104, 106, 108.

The train of cryogen pulses 112 that is delivered after the conclusion of the delivery of the power pulse 100 may range from two (2) milliseconds to 200 milliseconds with an adjustment increment of 1 millisecond. The maximum temperature measured by the sensors 52 and allowed to persist while delivering the power pulse 100 may range from 0° C. to 50° C. with an adjustment increment of 1° C. In response to this maximum temperature selection and during energy delivery, the system controller 18 may compare the selected maximum temperature with a median temperature determined from the readings supplied by the sensors 52. The sensors 52 may supply these readings at regular time intervals to the system controller 18. If the selected maximum temperature is exceeded and sustained over a given time period, corrective measures may be taken. For example, power delivery may be discontinued, the pulses 112 may be triggered for delivery, and an audible warning tone may be sounded.

In a preferred embodiment, the treatment electrode 20 is maintained stationary during over the entire duration of the treatment repetition and any perceived movement is unintentional. In this instance, the cryogen pulses 104, 106, 108, 110, 112 (or at least cryogen pulses 106, 108, 110) and treatment pulse 100 are delivered while the treatment electrode 20 is stationary.

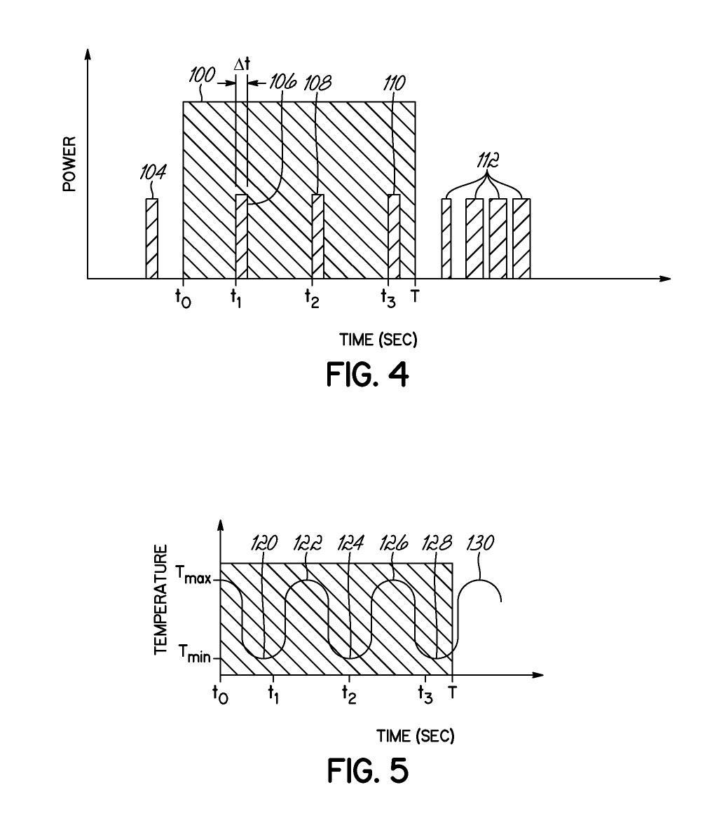

FIG. 5 diagrammatically shows changes in the temperature of the region 31 of the tissue 30 in response to the cryogen delivered by the protocol for a treatment repetition of FIG. 4 and is displayed with the same time scale as in FIG. 4. Region 31 is cooled by the initial cryogen pulse 104 with region 31 reaching a minimum temperature (Tₘᵢₙ) 120 shortly before cryogen pulse 106 at time t₁. Although the thermal mass is small so that the region 31 of tissue 30 is rapidly cooled, there is a short time delay before heat extracted from the region 31 by the cryogen pulse 106 begins to cool region 31. The region 31 heats to a maximum temperature (Tₘₐₓ) 122 as the effect of cryogen pulse 104 dissipates and before cryogen pulse 106 begins to cool the region 31. The region 31 cools to another minimum temperature (Tₘᵢₙ) 124 in the temperature curve in response to cryogen pulse 106. As heating continues and cooling dissipates, the region 31 again heats toward a maximum temperature (Tₘₐₓ) 126 in the temperature curve. In response to cryogen pulse 108 and after a short time delay relative to t₂, the region 31 cools toward another minimum temperature (Tₘᵢₙ) 128. Cryogen pulse 110 causes a similar effect and, in particular, causes region 31 to cool after rising to a maximum temperature (Tₘₐₓ) 130.

The oscillation in the tissue temperature of region 31 stimulates impulses communicated from nerves contained in the tissue of region 31 to the brain that mitigate the pain resulting from the tissue heating. The extent of the temperature change will lessen with greater depth relative to the skin surface 26 with shallow portions of region 31 at or near the skin surface 26 experiencing the largest range of temperature changes and deep portions of region 31 at or near the interface with region 32 experiencing the smallest range of temperature changes. The temperature in region 32 is substantially unaffected by the cooling and, instead, is primarily only heated by the electromagnetic energy.

The temperature of region 31 may vary in a sinusoidal manner or quasi-sinusoidally; however, the variation may exhibit a smooth repetitive oscillation or may lack the smoothness depicted in the representative embodiment. The temperature oscillation causes the tissue temperature of region 31 to vary between alternate extremes (the maximum temperature Tₘₐₓ and minimum temperature Tₘᵢₙ) over a definable period of time for the treatment repetition. The overlap of the tissue heating with the oscillating tissue cooling will tend to increase the temperature of the region 31 of tissue 30 such that the minimum and maximum temperatures of region 31 may increase with increasing time during energy delivery. In one embodiment, the minimum temperatures (Tₘᵢₙ) 120, 124, 128 of region 31 are approximately equal and/or the maximum temperatures (Tₘₐₓ) 122, 126, 130 of region 31 are approximately equal. The equivalent may be achieved by varying the duration and/or the amount of cryogen in the cryogen pulses 106, 108, 110. However, an increasing temperature of region 31 from the electromagnetic energy being delivered over increasing treatment time of a repetition may impart an inclining shift that ramps the envelope of the temperature profile toward higher tissue temperatures as a function of time (i.e., with increasing treatment time). In other words, minimum temperature (Tₘᵢₙ) 124 may be hotter than minimum temperature (Tₘᵢₙ) 120, minimum temperature (Tₘᵢₙ) 128 may be hotter than minimum temperature (Tₘᵢₙ) 124, maximum temperature (Tₘₐₓ) 126 may be hotter than minimum temperature (Tₘᵢₙ) 122, and minimum temperature (Tₘᵢₙ) 130 may be hotter than minimum temperature (Tₘᵢₙ) 126.

While FIG. 5 depicts the oscillation of the temperature of region 31 of tissue 30, a person having ordinary skill in the art will appreciate that the temperature of the treatment electrode 20 will likewise oscillate with variations between one or more minimum temperatures, similar or identical to minimum temperatures (Tₘᵢₙ) 120, 124, and 128, and one or more maximum temperatures, similar or identical to minimum temperatures (Tₘₐₓ) 122, 126, and 130.

In use to perform a treatment procedure, the physician selects a type of treatment tip 14 based on the procedure to be performed and the size of the surface area on the patient to be treated, as well as the depth of cooling and heating desired for the treatment procedure. After choosing the treatment tip 14 and attaching it to the handpiece 12, the physician marks the intended treatment area on the patient with a grid of removable markings that are easily wiped away post-procedure. Each discrete square in the grid corresponds approximately to the size of the portion 60 of the treatment electrode 20 that is placed in direct contact with the skin surface 26. The markings operate as a placement guide on the patient's skin surface 26 for the treatment procedure. The return electrode 56 is attached to the patient to close the current path for the high frequency current back to the generator 16.

After the optional application of a conductive fluid, each square within the grid is sequentially treated with high frequency energy delivered from the treatment electrode 20. Specifically, at each grid square, the physician lands the treatment electrode 20 directly against the patient's skin and actuates the activation button 54 on the handpiece 12. The treatment electrode 20 transmits high frequency energy to the tissue 30 beneath the skin surface 26 while serving as a contact cooling membrane for the cryogen. Information about skin temperature and contact, treatment force or pressure against the skin, cooling system function, and other types of relevant data, such as impedance may be supplied from the treatment tip 14 to the system controller 18 to precisely and safely control the high frequency energy and coolant delivery to each treatment site in the grid. Cooling region 31 limits the temperature to lessen the likelihood of thermal damage to the region 31, which may be the epidermis. While maintaining contact with the skin surface 26 during each repetition, power and cryogen are delivered according to the protocol shown in FIG. 4, or a different protocol consistent with the principles of the invention.

After energy delivery is completed during each repetition, the handpiece 12 is maneuvered to lift the portion 60 of the treatment electrode 20 from the skin surface 26. The handpiece 12 and treatment tip 14 are moved among subsequent treatment locations in the grid and energy is delivered in a similar manner using the protocol of FIG. 4 for treating large regions on the patient, such as the patient's face. Multiple passes over the entire grid of the treatment zone, separated in time by a quiescent period of a few minutes and using the protocol of FIG. 4, may be used to enhance the treatment, as is understood by persons skilled in the art. Multiple treatments, which are separated temporally by a lengthier healing period, may be needed for a successful treatment that supplies the desired cosmetic effect.

Delivering the cryogen in a series of pulses at each treatment zone and for each repetition is effective to decrease the sensation of pain experienced by the patient from the delivery of electromagnetic energy during a treatment procedure. The human nervous system is believed to be confused by the periodic modulation of the tissue temperature which results in a lower level of perceived pain, which allows for the delivery of higher amounts of high frequency energy while minimizing the associated pain perceived by the patient. Effectively, the acute pain during each repetition is converted into a form of tolerable, chronic, heat-related discomfort following the repetition.

References herein to terms such as “vertical”, “horizontal”, etc. are made by way of example, and not by way of limitation, to establish a frame of reference. It is understood that various other frames of reference may be employed for describing the invention without departing from the spirit and scope of the invention. It is also understood that features of the invention are not necessarily shown to scale in the drawings. Furthermore, to the extent that the terms “composed of”, “includes”, “having”, “has”, “with”, or variants thereof are used in either the detailed description or the claims, such terms are intended to be inclusive and open-ended in a manner similar to the term “comprising.”

It will be understood that when an element is described as being “attached”, “connected”, or “coupled” to another element, it can be directly connected or coupled to the other element or intervening elements may be present. In contrast, when an element is described as being “directly attached”, “directly connected”, or “directly coupled” to another element, there are no intervening elements present.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

While the invention has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. Thus, the invention in its broader aspects is therefore not limited to the specific details, representative apparatus and method, and illustrative example shown and described. Accordingly, departures may be made from such details without departing from the spirit or scope of applicants' general inventive concept.

What is claimed is:

1. A method of operating an apparatus to treat a patient with electromagnetic energy, the method comprising:
delivering the electromagnetic energy through a skin surface to heat a first region of tissue beneath the skin surface and to heat a second region of the tissue between the first region and the skin surface; and
during the delivery of the electromagnetic energy to the first and second regions, causing a temperature of the second region of the tissue to oscillate between at least one maximum temperature and at least one minimum temperature colder than the at least one maximum temperature.

2. The method of claim 1 wherein causing the temperature of the second region of the tissue to oscillate between the at least one maximum temperature and the at least one minimum temperature comprises:
while continuously heating the first and second regions with the electromagnetic energy, extracting thermal energy from the second region over intervals spaced apart in time to intermittently cool the second region.

3. The method of claim 2 wherein the extraction of the thermal energy occurs a given frequency effective to reduce pain associated with the delivery of the electromagnetic energy.

4. The method of claim 2 wherein the extraction of the thermal energy is periodic at a given frequency effective to reduce pain associated with the delivery of the electromagnetic energy.

5. The method of claim 1 wherein the oscillation of the temperature occurs at a given frequency effective to reduce pain associated with the delivery of the electromagnetic energy.

6. The method of claim 1 wherein the electromagnetic energy is delivered from a treatment electrode, and delivering the electromagnetic energy comprises:
contacting a portion of the treatment electrode with the skin surface; and
while maintaining the contact between the portion of the treatment electrode and the skin surface, delivering the electromagnetic energy.

7. The method of claim 6 wherein causing the temperature of the second region of the tissue to oscillate between the at least one maximum temperature and the at least one minimum temperature comprises:
supplying a plurality of discrete amounts of a cryogen in a timed sequence as a series of cryogen pulses effective to intermittently cool the treatment electrode; and
cooling the second region by heat transferred from the second region through the skin surface to the portion of the treatment electrode in contact with the skin surface.

8. The method of claim 7 wherein the timed sequence for the cryogen pulses is selected to periodically cool the treatment electrode at a given frequency effective to reduce pain associated with the delivery of the electromagnetic energy.

9. The method of claim 7 wherein the cryogen pulses are supplied in an overlapping temporal relationship with the delivery of the electromagnetic energy.

10. The method of claim 7 wherein at least one of the cryogen pulses is delivered before the delivery of the electromagnetic energy begins, and at least one of the cryogen pulses is delivered during the delivery of the electromagnetic energy.

11. The method of claim 7 wherein the portion of the treatment electrode and the skin surface are sustained in a stationary contacting relationship while the electromagnetic energy is delivered through the skin surface to the tissue and the series of cryogen pulses are supplied to cool the portion of the treatment electrode.

12. The method of claim 1 wherein the at least one maximum temperature includes a plurality of maximum temperatures that increase with increasing time during the delivery of the electromagnetic energy to the first and second regions, or the at least one minimum temperature includes a plurality of minimum temperatures that increase with increasing time during the delivery of the electromagnetic energy to the first and second regions.

13. A method of operating an apparatus having a treatment electrode, the method comprising:
delivering electromagnetic energy from the treatment electrode; and
during the delivery of the electromagnetic energy, causing a temperature of the treatment electrode to oscillate between at least one maximum temperature and at least one minimum temperature colder than the at least one maximum temperature.

14. The method of claim 13 wherein causing the temperature of the treatment electrode to oscillate between the at least one maximum temperature and the at least one minimum temperature comprises:
supplying a plurality of discrete amounts of a cryogen in a timed sequence as a series of cryogen pulses that intermittently cool the electrode.

15. The method of claim 14 wherein the cryogen pulses are timed to periodically cool the treatment electrode at a given frequency.

16. The method of claim 14 wherein the cryogen pulses are supplied in an overlapping temporal relationship with the delivery of the electromagnetic energy.

17. A system for transcutaneously treating tissue located beneath a skin surface with electromagnetic energy, the system comprising:
a cryogen supply configured to contain a cryogen;
a handpiece including a valve coupled in fluid communication with the cryogen supply;
a treatment tip attached in a removable manner with the handpiece, the treatment tip including a treatment electrode configured to deliver the electromagnetic energy through the skin surface to the tissue and a nozzle coupled in fluid communication with the valve in the handpiece;
a generator configured to generate the electromagnetic energy, the generator electrically coupled by the handpiece with the treatment electrode; and
a controller connected in electrical communication with the generator and with the valve, the controller configured to cause the generator to deliver the electromagnetic energy to the treatment electrode for delivery through the skin surface to the tissue and configured to trigger the valve to deliver a plurality of discrete pulses each containing an amount of the cryogen to the nozzle during delivery of the electromagnetic energy.

18. The system of claim 17 wherein the nozzle is configured to deliver the cryogen to the treatment electrode, and the treatment electrode includes a portion configured to contact the skin surface.

19. The system of claim 17 wherein the controller is configured to trigger the valve at a series of times to deliver the plurality of discrete pulses of the cryogen from the nozzle during delivery of the electromagnetic energy.
